(19) United States
(12) Patent Application Publication
(10) Pub. No.: US 2004/0091982 A1
(43) Pub. Date: May 13, 2004

(54) Isocyanate free polyurethane production process via carbamate polyesterification

(76) Inventors: Norman Gee, Accrington (GB); Alan Taylor, Accrington (GB)

Correspondence Address: NIXON & VANDERHYE, PC, 1100 N GLEBE ROAD, 8TH FLOOR, ARLINGTON, VA, 22201-4714, US

(21) Appl. No.: 10/466,611

(22) PCT Filed: Jan. 16, 2002
(86) PCT No.: PCT/GB02/00189

(30) Foreign Application Priority Data
Jan. 19, 2001 (GB) 0101464.6

(51) Int. Cl. C12P 13/00

(52) U.S. Cl. 435/128

(57) ABSTRACT

A process for producing a polyurethane, comprising polyesterifying a carbamate containing at least two functional groups selected from hydroxyl groups and carboxylic acid groups or esters or anhydrides thereof, in the presence of a polyesterification enzyme, and optionally in the presence of one or more copolymerizable monomers having two or more functional groups selected from hydroxyl groups and carboxylic acid groups or esters or anhydrides thereof. This process enables a broader range of polyurethanes to be accessed commercially than the previously described processes.

### FIELD OF THE INVENTION

The present invention relates to a new process for producing polyurethanes and to novel polyurethanes.

### BACKGROUND OF THE INVENTION

Polyester based polyurethanes are well known and used widely for many applications, including thermoplastic polyurethanes, surface coatings, textile coatings, adhesives, elastomers, polyurethane foams and polyurethane dispersions.

These materials are manufactured from polyester resins, typically produced by reacting difunctional alcohols and difunctional acids, followed by reaction with a diisocyanate to produce a polyurethane. Many diols and diacids are currently available. For example, diethylene glycol, ethylene glycol, 1,4-butanediol, 1,6-hexanediol and neopentylglycol are typically used along with adipic acid, succinic acid, terephthalic acid and many other diacids.

Polyurethane grade polyesters are produced with low water content, typically less than 0.05%, a low final acid value, typically less than 2 mg KOH/g and to a hydroxyl value specification. Hydroxyl values can vary from around 10 to 225 mg KOH/g depending on the molecular weight of the polyester produced.

The resulting hydroxyl functional polyesters can be reacted with di- or tri-functional isocyanates in order to produce polyurethanes. Isocyanates which are currently commercially available include toluene diisocyanate, 4,4′-diphenylmethane diisocyanate, 1,6-hexamethylene diisocyanate and its derivatives, isophorone diisocyanate and its derivatives, 4,4′-dicyclohexylmethane diisocyanate and tetramethylxylene diisocyanate. The polyurethanes produced can be prepolymers with isocyanate functionality, or alternatively hydroxyl functional polyester based polyurethanes can be produced by using an excess of polyester during the reaction. Thermoplastic polyurethanes can also be produced. However, the number of commercially available isocyanates is a limitation to the formulation of these materials.

A new process is therefore desired which enables a broader range of polyurethanes to be produced from commercially available starting materials.

### SUMMARY OF THE INVENTION

The present inventors have developed a new technology which allows polyester based polyurethanes to be manufactured without involving isocyanate reagents, that is the production of a “non-isocyanate polyurethane (NIPU)”. The process of the present invention uses carbamates as the source of nitrogen in the urethane linkage of the polyurethane products. This in turn allows readily available diamines (which are reacted with a cyclic carbonate in an initial reaction) to be used as starting materials for a polymerisation reaction to produce the desired polyurethane.

This strategy reverses the conventional stepwise methodology, whereby the urethane linkage is always introduced after the major polyesterification process is effected. The conventional order of reaction is dictated by the fact that urethane linkages are susceptible to thermal dissociation at temperatures above 150° C.; hence conventional polyesterification (which requires temperatures of 200 to 220° C. to force the reaction to completion) of urethane-group-containing precursors would inevitably lead to discolouration of the product and breakdown of the urethane linkage, with consequent loss of fidelity of the structure and possibly free isocyanate being left in the finished product. The present inventors have established that enzyme-catalysed polymerisation enables the polymerisation to be carried out effectively at less than 100° C., preventing thermal degradation of the urethane groups.

The use of the process of the present invention thus provides a low temperature route to already known materials. It further enables a broader range of polyurethanes to be manufactured than is possible using the isocyanate route since certain diisocyanates, such as ethylene diisocyanate, are not commercially available or easily synthesised whereas the corresponding diamine precursors can be obtained economically. For instance, compounds such as ethylene diamine, which provides the same polyurethane as would be produced via the isocyanate route using ethylene diisocyanate, are generally easy to obtain in bulk. Avoiding use of isocyanates which are highly dangerous is also advantageous for environmental reasons and to simplify handling procedures.

The provision of a broader range of polyurethanes will, in turn, provide the skilled person in the art with access to polymers with new and different combinations of properties. This may lead to improvements in the currently known applications such as coatings and adhesives and may further lead to new applications for polyurethanes.

### DETAILED DESCRIPTION OF THE INVENTION

Accordingly, the present invention provides a process for producing a polyurethane, comprising polyesterifying at least one carbamate, for example an aliphatic monocarbamate and/or an aliphatic or aromatic biscarbamate and/or an aliphatic or aromatic polycarbamate, said carbamate containing at least two functional groups selected from hydroxyl groups and carboxylic acid groups or esters or anhydrides thereof, in the presence of a polyesterification enzyme, and optionally in the presence of one or more copolymerizable monomers having two or more functional groups selected from hydroxyl groups and carboxylic acid groups or esters or anhydrides thereof.

Polymerisation

The-process of the present invention comprises the enzyme-catalysed polyesterification of a carbamate, for example an aliphatic monocarbamate and/or an aliphatic or aromatic biscarbamate and/or an aliphatic or aromatic polycarbamate, the carbamate containing at least two functional groups selected from hydroxyl groups and carboxylic acid groups or esters or anhydrides thereof. Hereinafter, unless indicated otherwise, a carbamate is an aliphatic monocarbamate, an aliphatic or aromatic biscarbamate or an aliphatic or aromatic polycarbamate, such as a tricarbamate.

Suitable aliphatic monocarbamates include those of formula (I):

R¹—O—CO—NH—R² (I)

wherein

R¹ is a C₁ to C₁₂ hydroxyalkyl group optionally having one or more carbon-carbon double bonds and optionally having one or more carbon-carbon triple bonds, R¹ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl) and

R² is a C₁ to C₁₂ alkyl group optionally having one or more carbon-carbon double bonds and optionally having one or more carbon-carbon triple bonds, R² being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl) and, when substituted by hydroxyl, may be the same as or different from R¹.

Examples of typical hydroxyalkyl groups R¹ include hydroxyethyl, hydroxypropyl, hydroxybutyl, hydroxyhexyl and hydroxyoctyl, each of which preferably has the hydroxyl group on the terminal carbon atom.

Preferably R² is an alkyl group with substituents such as a carboxylic acid group or ester or anhydride thereof, or alternatively hydroxyl groups.

Suitable aliphatic or aromatic biscarbamates include those of formula (II):

R⁵—O—CO—NH—R—NH—CO—O—R⁶ (II)

wherein R⁵ and R⁶, which may be identical or different, are hydroxyalkyl groups as defined above for R¹ and R is a single bond, an aromatic group, a cycloaliphatic group or a C₁ to C₁₂ alkylene group optionally having one or more carbon-carbon double bonds and optionally having one or more carbon-carbon triple bonds, R being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl).

Suitable aromatic and cycloaliphatic groups for use as the group R include phenylene, toluene such as 2,4-toluene or 2,6-toluene, naphthylene, dianisidine, 4,4′-methylene-bis(phenyl), 2,4′-methylene-bis(phenyl), 4,4′-ethylene-bis(phenyl), ω,ω′-1,3-dimethyl benzene, ω,ω′-1,4-dimethyl benzene, ω,ω′-diethyl benzene, ω,ω′-dimethyl toluene, ω,ω′-diethyl toluene, cyclohexylene, ω,ω′-1,4-dimethyl cyclohexane, ω,ω′-1,3-dimethylcyclohexane, 1-methyl-2,4-cyclohexylene, 4,4′-methylene-bis (cyclohexyl), dimer acid, 1,4-bis-(prop-2yl) benzene and 1,3-bis(prop-2yl) benzene, the latter two compounds being attached to the remainder of the biscarbamate group at the 2-position on each propyl moiety.

Suitable C₁ to C₁₂ alkylene groups for use as the group R include methylene, ethylene, propylene, butylene, hexamethylene, octamethylene, decamethylene, dodecamethylene or 2,4,4-trimethyl hexamethylene, each of which may be unsubstituted or substituted.

Suitable aliphatic or aromatic polycarbamates include those of formula (IIA):

R^{a}(—NH—CO—O—R^{5a})ₘ (IIA)

wherein each R^{5a}, which may be identical or different, is a hydroxyalkyl group as defined above for R¹; R^{a} is an aromatic group, a cycloaliphatic group or an alkylene group as defined above for R; and m is an integer of 3 or more, preferably 3 or 4.

The hydroxyl groups present on the carbamate, or on any of the other monomers present, are preferably non-sterically hindered. Tertiary and sterically hindered primary and secondary hydroxyls are unlikely to react under the conditions of the enzyme catalysed process. Variation in the reaction conditions, such as the specific enzyme used or the solvent present, can however affect the degree of steric hindrance which can be tolerated in the functional groups of the carbamate.

The degree of steric hindrance which can be tolerated under specified reaction conditions, whilst still allowing polymerisation to proceed, can be determined by a simple trial and error technique. For example, a carbamate diol containing a potentially sterically hindered hydroxyl group may be polymerised with a diacid which is known to polymerise under the selected reaction conditions, for example adipic acid. If the simple polymerisation does not proceed, the degree of steric hindrance is too great for use in the present invention.

Similarly, the carboxylic acid groups, or esters or anhydrides thereof which are present on the carbamate or on any of the other monomers present are preferably non-sterically hindered groups. The degree of steric hindrance which can be tolerated can be determined by trial and error in a similar manner to that described above.

The carbamates of the invention preferably contain two non-sterically hindered hydroxyl groups, one non-sterically hindered hydroxyl group and one non-sterically hindered carboxylic acid group or an ester or anhydride thereof, or two non-sterically hindered carboxylic acid groups or esters or anhydrides thereof.

The carbamates may also optionally contain further substituents, such as further hydroxyl groups or carboxylic acid groups or esters or anhydrides thereof. When such further substituents are present, cross-linking may occur during the polymerisation reaction. The ability to introduce branching into the resin in this way enables a much wider variety of polyurethanes to be accessed.

Particularly preferred monocarbamates include those derived from an aminol or amino acid, or ester thereof, and ethylene carbonate. Particularly preferred biscarbamates include those derived from primary diamines, especially hexamethylene diamine and two molecules of ethylene carbonate. Particularly preferred polycarbamates are tricarbamates such as those derived from melamine and three molecules of ethylene carbonate.

The polyesterification of the invention can be carried out without the presence of any further, non-carbamate monomers if the carbamate monomer has at least one hydroxyl group and at least one carboxylic acid group or an ester or anhydride thereof, or if two or more carbamate monomers are present which may be copolyesterified. Examples of polyesterifications which can be carried out without the presence of non-carbamate monomers include the homopolymerization of a hydroxy-carboxy carbamate; the copolymerisation of a dihydroxy carbamate with a dicarboxy carbamate; or the terpolymerisation of a dihydroxy carbamate with a dicarboxy carbamate and a hydroxy-carboxy carbamate.

When the carbamate monomers are (co)polyesterifiable, one or more monomers may, if desired, be present in addition to the carbamate(s), which monomers are copolymerizable with the carbamate(s). Each additional monomer has at least two functional groups selected from hydroxyl groups and carboxylic acid groups or esters or anhydrides thereof. Copolymerizable monomers include any monomers which will polymerise with the carbamate(s) under the polymerisation conditions used. To determine whether a monomer is copolymerizable under the specified conditions and with the specified carbamate(s), trial and error may be used.

Typically, the polyesterification is carried out in the presence of one or more monomers selected from aliphatic dicarboxylic acids or esters or anhydrides thereof, aliphatic hydroxycarboxylic acids or esters or anhydrides thereof, together with a diol or polyol.

Suitable aliphatic dicarboxylic acids include those of formula (III):

HO₂C—R⁷—CO₂H (III)

wherein R⁷ is a bond or a C₁ to C₁₂ alkylene group optionally having one or more carbon-carbon double bonds and optionally having one or more carbon-carbon triple bonds, R⁷ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl).

Aliphatic hydroxycarboxylic acids suitable for use in this process include those of formula (IV):

HOCH₂—R⁸—CO₂H (IV)

wherein R⁸ is as defined for R⁷ above.

Suitable aliphatic diols include those of formula (V):

HOCH₂—R⁹—CH₂OH (V)

wherein R⁹ is as defined for R⁷ above.

Suitable aliphatic polyols include those of formula (VI):

HOCH₂—R¹⁰—CH₂OH (VI)

wherein R¹⁰ is a C₁ to C₁₂ hydroxyalkylene group optionally having one or more carbon-carbon double bonds and optionally having one or more carbon-carbon triple bonds, R¹⁰ being optionally further substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl).

Each of the C₁ to C₁₂ alkyl groups, alkylene, hydroxyalkyl and hydroxyalkylene groups mentioned herein may be unsubstituted, substituted or, in the case of hydroxyalkyl and hydroxyalkylene groups, further substituted, and may be cyclic, branched or straight chain, optionally having at least one carbon-carbon double bond, either in the cis- or trans-conformation and optionally having at least one carbon-carbon triple bond. When the C₁ to C₁₂ alkyl, alkylene, hydroxyalkyl or hydroxyalkylene group has more than one double or triple carbon-carbon bond, these bonds may be conjugated or non-conjugated. The C₁ to C₁₂ alkyl, alkylene, hydroxyalkyl, or hydroxyalkylene groups are optionally (further) substituted with one or more substituents which, when there are two or more substituents, may be the same or different. Halogen substituents are preferably fluorine, chlorine or bromine.

The polyols of formula (VI) used herein have at least three hydroxyl groups, preferably 3, 4 or 5 hydroxyl groups. Suitable polyols include trimethylolpropane, pentaerythritol and triols, especially glycerol. The presence of the third hydroxyl group may introduce branching into the polyurethane. Use of glycerol generally results in a linear polymer when using the lipase from Candida antarctica (see below) as this enzyme preferentially esterifies the primary hydroxyls, but branched products may be obtained using certain enzymes.

Preferably the diol of formula (V) has from 2 to 14 carbon atoms and is suitably an α,ω-diol, for example 1,4-butanediol, diethylene glycol, ethylene glycol, propylene glycol, pentanediol, hexane-1,6-diol or dodecane-1,12-diol, most preferably 1,4-butanediol.

Preferably the hydroxycarboxylic acids of formula (V) have from 2 to 14 carbon atoms and are hydroxy-straight chain aliphatic carboxylic acids. Examples of suitable hydroxy acids include glycolic acid, lactic acid, 2-hydroxy butyric acid, 2-hydroxy isobutyric acid, 2-hydroxy caproic acid, 2-hydroxy isocaproic acid, citric acid or malic acid.

At high dilution certain hydroxy carboxylic acids of formula (IV) tend to form lactones and it is therefore preferred that, when such hydroxy acids are used in the enzyme catalysed process, they are used only in high concentration in order to avoid the unwanted lactonisation reaction.

Preferably the diacid of formula (III) has from 2 to 14 carbon atoms, and is suitably an α,ω-diacid, for example, oxalic acid, succinic acid, fumaric acid, citric acid, malic acid, malonic acid, maleic acid or adipic acid.

An ester of a diacid of formula (III) may be a monoester or a diester, for example a mono or dialkyl ester. Preferably the alkyl groups of an alkyl ester of a diacid or an alkyl ester of a hydroxy acid are each of 1 to 4 carbon atoms, and more preferably the derivative is a methyl or ethyl ester or diester, most preferably methyl adipate or dimethyl adipate.

Owing to the low temperatures used in the enzyme catalysed process compared with those of conventional chemically catalysed polyesterifications, it is possible to use diacids and hydroxyacids, such as oxalic acid, lactic acid and glycolic acid, which decarboxylate at elevated temperatures and it is thereby possible to produce a broader range of polyurethanes.

Preferably, the hydroxyl groups present on the hydroxy acids and the diols, and at least two of the hydroxyl groups present on the polyols, are primary or secondary hydroxyl groups, more preferably non-sterically hindered hydroxyl groups. It is also preferred that the carboxylic acid groups or esters or anhydrides thereof which are present on the diacids or the hydroxyacids are non-sterically hindered. As discussed with reference to the carbamates, tertiary hydroxyl groups or sterically hindered functional groups tend not to react under enzyme catalysed polymerisation conditions, although their reactivity varies depending on the particular conditions chosen. Trial and error may be used to determine the degree of steric hindrance that can be tolerated under any specific conditions.

When the carbamate monomers are not (co)polyesterifiable when used without additional monomers, other monomers of formulae (III), (IV), (V) or (VI) above will be used. Thus when the carbamate(s) used contain only hydroxyl groups and do not contain carboxylic acid groups or esters or anhydrides thereof, at least one aliphatic dicarboxylic acid or an ester or anhydride thereof must be present in the polymerisation reaction. Similarly, when the carbamate(s) used contain only carboxylic acid groups or esters or anhydrides thereof and do not contain any hydroxyl groups, at least one aliphatic diol or polyol must be present in the polymerisation reaction.

In the various embodiments of the present invention, the combination of monomers which are used in the polymerisation process in addition to the carbamates may include (subject to the requirements mentioned above) diacid alone; hydroxy acid alone; diacid and diol; diacid and polyol; diacid, diol and polyol; diacid, hydroxy acid and diol; diacid, hydroxy acid and polyol; hydroxy acid and diol; hydroxy acid and polyol; polyol alone or diol alone, or any other suitable combination of monomers, for example where the diacid is replaced by its methyl ester or ethyl ester derivative.

Preferred combinations of monomers include a dihydroxy carbamate with a diacid or a dimethyl ester of a diacid, such as adipic acid or dimethyladipate; a dihydroxy carbamate with a diacid or a dimethyl ester of a diacid, and a diol, such as adipic acid/1,4-butanediol, dimethyladipate/1,4-butanediol, adipic acid/diethylene glycol, dimethyladipate/diethylene glycol, adipic acid/1,6-hexanediol or dimethyladipate/1,6-hexanediol; a dihydroxy carbamate with a diacid or a dimethyl ester of a diacid and a polyol, such as adipic acid/glycerol or adipic acid/trimethylolpropane; a dihydroxy carbamate with a diacid or a dimethyl ester of a diacid, a diol and a polyol, such as adipic acid/1,4-butanediol/glycerol or adipic acid/1,4-butanediol/trimethylolpropane; a hydroxy-carboxy carbamate with a diacid or a dimethyl ester of a diacid and a diol, such as adipic acid/1,4-butanediol, dimethyladipate/1,4-butanediol, adipic acid/diethylene glycol or adipic acid/1,6-hexanediol; a dicarboxy carbamate with a diol, such as 1,4-butanediol, 1,6hexanediol or diethylene glycol; a dicarboxy carbamate with a polyol, such as glycerol or trimethylolpropane; or a dicarboxy carbamate with a diacid or a methyl ester of a diacid and a diol, such as adipic acid/1,4-butanediol, dimethyladipate/1,4-butanediol, adipic acid/diethylene glycol or adipic acid/1,6-hexanediol.

The enzymatically polyesterifiable carboxylic acid groups and enzymatically polyesterifiable hydroxyl groups of the reactants are generally present in substantially equal numbers. The reaction may be carried out with a stoichiometric imbalance, but this generally results in a product having a lower weight average molecular weight than if the reactants are used in equimolar amounts.

Typically, the carbamate has two hydroxyl groups as its functional groups if it is a mono- or biscarbamate, three hydroxyl groups as its functional groups if it is a tricarbamate, or further hydroxyl groups if it is a polycarbamate, and the other monomers present are a diacid and a diol.

The process of the invention may be used to produce cross-linked polyurethanes. If cross-linking is desired, additional functional groups, i.e. at least three functional groups (such as 3, 4 or 5 functional groups) selected from hydroxyl groups and carboxylic acid groups or esters or anhydrides thereof, must be present on at least one of the monomers. For example, the carbamate may be a polycarbamate containing at least one functional group on each carbamate chain, or a mono- or biscarbamate which contains three such functional groups, for example three hydroxyl groups. Alternatively, or additionally, a polyol, a hydroxydiacid, a dihydroxy acid and/or a triacid may be present as a comonomer. Two or more monomers containing three or more functional groups may be used if desired. Preferably at least three of the three or more functional groups present on each monomer are non-sterically hindered groups which, if they are hydroxyl groups, are preferably primary or secondary groups. Further cross-linking may be introduced as a subsequent step after the enzymatic polyesterification using traditional chemical methods to cross-link at branch points introduced by use of such tri- or higher-functional monomers.

The reaction may be carried out without the use of a solvent, or an organic solvent may be present. Suitable solvents are inert to the reaction, do not inactivate the enzyme and are sufficiently immiscible with water to prevent dehydration of the enzyme. Certain aromatic solvents are suitable, such as toluene.

The enzymes which may be used in the present invention include commercially available lipases. When the polyesterification is carried out in the absence of a solvent the preferred enzyme is the lipase derived from Candida antarctica.

Other suitable lipases can be identified by simple trial and error experimentation. For further details of the enzymes and substrates which may be used, and of enzyme catalysed processes, see GB-A-2272904, PCT/GB93/02461 and PCT/GB97/01084. Each of these documents is incorporated herein by reference.

Other enzymes capable of polyesterification of the foregoing monomers to form polyurethanes may readily be identified by simple trial and error experimentation.

The enzyme used in the present process may be bound on an inert carrier, for instance a polymer such as an anion exchange resin, an acrylic resin or a polypropylene, polyester or polyurethane resin or may be used in free form. When the enzyme is bound on an inert carrier it can easily be removed from the reaction mixture without the need for complicated purification steps. This enables the enzyme to be recovered and re-used. A preferred example is the lipase available from Novo Industri AS, Novozyme 435™, where the enzyme is immobilised on a macroporous acrylic resin. Alternatively a free enzyme may be used, which is left in the polymeric product. In this case deactivation of the enzyme may be desirable at a later stage in the process. A solution of a free lipase, for example Novozyme 525™, is preferred.

The total reaction time is typically from 3 to 5 days, preferably from 3 to 4 days.

The activity of the enzyme may be affected by materials present in the reaction mixture, for example the lipase from Candida antarctica is inhibited by glycerol. It is preferable not to include branched polyfunctional monomers, particularly secondary alcohols, in the initial reaction mixture, but to delay their addition until after the reaction is started to avoid reducing enzyme activity. If a branched polyfunctional monomer is added to the reaction mixture at least 12 hours, for example at least 14 hours, 16 hours or 24 hours after the start of the reaction, when the enzyme is still present in the reaction mixture, the enzyme activity will be reduced, but not completely, and the reaction will continue at a slower rate than if the branched polyfunctional monomer had not been added.

The amount of enzyme used is not critical and is generally limited by economic considerations. Too little enzyme will result in a slow reaction whereas too much enzyme simply increases costs unnecessarily. With the lipase derived from Candida antarctica, it has been found convenient to use from 0.1 to 1.5% by weight of supported enzyme (calculated as the weight of enzyme) based on the total weight of monomers (including carbamate) present, preferably 0.1 to 0.6% and most preferably 0.15 to 0.3% of enzyme.

The process is generally carried out at a temperature of from 10 to 100° C., preferably from 40 to 70° C. Above 100° C., most enzymes will denature but enzymes may be used which have a denaturation temperature higher than 100° C. and then the reaction may be carried out at a higher temperature (subject to the stability of other reagents). Below 10° C. the reaction is very slow and takes an uneconomically long time to go to completion.

The process is generally carried out at atmospheric or reduced pressure. The water produced by the reaction is generally removed during the reaction, conveniently by reducing the pressure during the course of the reaction. For example, the pressure may be reduced to from 1×10³ to 3×10⁴ Pa (10 to 300 mbar), preferably to about 5×10³ Pa to 1×10⁴ Pa (50 to 100 mbar). Typically, the pressure is reduced in stepwise manner throughout the reaction. For example, an initial pressure of 2.5×10⁴ to 3×10⁴ Pa (250 to 300 mbar) may be applied, being then reduced to 1×10⁴ to 1.5×10⁴ Pa (100 to 150 mbar) and then further to 1×10³ to 5×10³ Pa (10 to 50 mbar). Alternatively the water may be removed with a wiped film evaporator under reduced pressure, for instance 500 or even 100 Pa or less (5 or 1 mbar or less).

Preparation of Carbamates

Typically, the carbamates used in the process of the present invention are prepared by reacting an aliphatic carbonate or hydroxy carboxylic acid or an ester or anhydride thereof, with a primary or secondary aminol, amino acid or ester thereof (to make monocarbamates), hydrazine or a diamine (to form biscarbamates) or a polyamine (to form polycarbamates). However, alternative routes may be used if desired.

Suitable aliphatic carbonates include those of formula (VII):

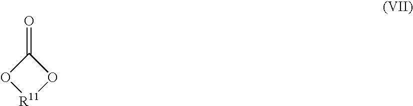

wherein R¹¹ is a C₂ to C₁₂ alkylene group optionally having one or more carbon-carbon double bonds and optionally having one or more carbon-carbon triple bonds, R¹¹ being unsubstituted or substituted with one or more substituents. Substituents on R¹¹ may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl). Alternatively the substituents themselves may bear one or more cyclic carbonate groups, such as the structures disclosed by Steblyanko et al, J. Polymer Sci., Part A, Polymer Chem., Vol. 38, pages 2375-2380 (2000), which document is incorporated herein by reference. Preferred groups R¹¹ include ethylene, propylene, butylene, hexylene and octylene.

Preferably the carbonates are C₂ to C₆ carbonates such as ethylene carbonate, propylene carbonate, butylene carbonate or hexamethylene carbonate.

Suitable aliphatic hydroxy carboxylic acids or esters or anhydrides thereof, are those of formula (VIII):

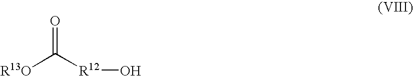

wherein R¹² is a C₂ to C₁₂ alkylene group optionally having one or more carbon-carbon double bonds and optionally having one or more carbon-carbon triple bonds, R¹² being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl) and R¹³ is hydrogen, or (CO)ₘR¹⁴ wherein m is 0 or 1 and R¹⁴ is C₁ to C₁₂ alkyl. Preferably the compound of formula (VIII) is 2-hydroxypropanoic acid.

Suitable aliphatic primary or secondary aminols, amino acids and esters thereof include those of formula (IX):

HN(R¹⁵)—R¹⁶—X (IX)

wherein

R¹⁵ is a C₂ to C₁₂ alkyl group optionally having one or more carbon-carbon double bonds and optionally having one or more carbon-carbon triple bonds, R¹⁵ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl),

R¹⁶ is a C₂ to C₁₂ alkylene group optionally having one or more carbon-carbon double bonds and optionally having one or more carbon-carbon triple bonds, R¹⁶ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl) and

X is a hydroxyl, carboxylic acid or ester group.

Preferably the aminols, amino acids or esters contain from 2 to 12 carbon atoms and the amine group is typically a primary amine. Preferred aminols include ethanolamine, 3-aminopropanol, 4-aminobutanol, 6-aminohexanol and 8-aminooctanol. Preferred amino acids or esters include glycine, 4-aminobutanoic acid, and 6-aminohexanoic acid and their methyl or ethyl esters.

Suitable primary or secondary aliphatic or aromatic diamines include those of formula (X):

HN(R¹⁸)—R ¹⁷—NH(R¹⁹) (X)

wherein

R¹⁷ is a bond or a group R as defined above,

R¹⁸ and R¹⁹ are the same or different and each is a C₁ to C₁₂ alkyl group optionally having one or more carbon-carbon double bonds and optionally having one or more carbon-carbon triple bonds, R¹⁸ and/or R¹⁹ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl).

Preferably the diamines are C₂ to C₁₂ primary diamines, which may be unsubstituted or substituted. Preferably the diamines are unsubstituted or substituted with hydroxyl groups or carboxylic acid groups or esters or anhydrides thereof. If such substituents are present on the alkylene chain of the diamine, this enables cross-linking to occur in the subsequent polymerisation reaction. Examples of suitable diamines include ethylene diamine, propylene diamine, butylene diamine, 1,6hexamethylene diamine and isophorone diamine.

Suitable primary or secondary aromatic or aliphatic polyamines are those of formula (XI)

R²⁰(—NHR²¹)ᵣ (XI)

wherein R²⁰ is a group R as defined above, each R²¹, which are the same or different, is a group R¹⁸ as defined above, and r is an integer of at least 3, preferably r is an integer m as defined above. Preferably the polyamines are triamines, such as melamine.

Suitable combinations of carbonates and diamines or aminols, amino acids or esters include ethylene diamine/ethylene carbonate; 1,6-hexamethylene diamine/ethylene carbonate; isophorone diamine/ethylene carbonate; propylene diamine/ethylene carbonate; 1,6-hexamethylene diamine/propylene carbonate; isophorone diamine/propylene carbonate; ethanolamine/ethylene carbonate; propanolamine/ethylene carbonate; ethanolamine/propylene carbonate; propanolamine/propylene carbonate; melamine/ethylene carbonate and melamine/propylene carbonate.

When the carbonate is reacted with a diamine, the molar ratio of carbonate to diamine is generally about 2:1. When the carbonate is reacted with a triamine, the molar ratio of carbonate to triamine is generally about 3:1.

The reactions between an aminol, amino acid or ester thereof and carbonate to form a monocarbamate and between a diamine and a carbonate to form a biscarbamate and between a polymine and a carbonate to form a polycarbamate are well known in the art. Any suitable techniques known in the art may therefore be used to carry out the reaction. Generally, the reaction is carried out at an elevated temperature, for example from 30 to 100° C., preferably from 40 to 70° C.

No solvent is required for this reaction, but an inert solvent may be used. Alternatively, the reaction may be carried out in the diol which is to be used as a monomer in the polymerisation reaction. The carbamate is produced as a solution in the diol and the polymerisation reaction can be carried out directly on this solution, without the need for purification of the carbamate, i.e. in a “one-pot” process.

If the carbamate is produced in a solvent other than the desired diol, or no solvent is used, the product may be recrystallised before being transferred to the polymerisation step.

In a particular aspect the present invention relates to a process as described above comprising the steps of:

(i) reacting a diamine with a carbonate to form a biscarbamate;

(ii) polyesterifying the biscarbamate so produced, in the presence of a polyesterification enzyme, with a diol and a diacid, to produce a polyurethane.

In an alternative aspect the present invention relates to a process as described above comprising the steps of:

(i) reacting a polyamine with a carbonate to form a polycarbamate;

(ii polyesterifying the polycarbamate so produced, in the presence of a polyesterification enzyme, with a diol and a diacid, to produce a polyurethane.

Polyurethanes

As used herein, the term ‘polyurethane’ encompasses materials obtainable by the process of the invention as described above.

The polyurethanes produced by the process of the present invention may be used as adhesives such as hot melt adhesives, textile coatings, surface coatings, thermoplastic polyurethanes, elastomers and polyurethane dispersions. Hydrophilic polyurethanes may also be produced by the present process, such compounds being suitable for use in polyurethane breathable textile coating. As has been described above, the polyurethanes may be cross-linked, providing more extensive applications of the compounds produced by the present invention.

Polyurethane compositions may be formed by mixing the polyurethanes of the present invention with other additives such as those conventionally present in adhesives or coatings such as antioxidants and catalysts. These compositions, or the polyurethanes themselves, may be moulded or otherwise formed into various shaped articles, such as coatings, which are suitable for the relevant application of the polyurethane or composition.

The polyurethanes of the invention preferably have a molecular weight of at least 1500, more preferably at least 2000.

The present invention will be described in more detail below with reference to the Examples.

### EXAMPLES

### Example 1A

| Materials | |
|---|---|
| Ethylene carbonate | 107 g |
| Ethylene diamine | 35.26 g |
| Toluene | 40 g |

Procedure

Ethylene carbonate was added to a flask and heated to 50° C. Ethylene diamine was added via a dropping funnel such that the temperature held at about 60° C. An initial exotherm was observed and toluene was added in order to reduce viscosity and help with heat transfer. Once the addition of the ethylene diamine was complete, the reaction mixture was held at 65° C. for 4 hrs.

The biscarbamate product was recrystallised in ethanol, washed and dried. The recorded yield was 60% theoretical. The melting point was determined as 93° C.

### Example 1B

| Materials | |
|---|---|
| Biscarbamate produced according to Example 1A | 15.0 g |
| 1,4-Butane diol | 22.5 g |
| Adipic Acid | 36.5 g |
| Novozyine 435 ™ | 1.49 g |

Procedure

A biscarbamate produced according to Example 1A was dissolved in the 1,4-butane diol at 70° C. followed by the addition of adipic acid (5 g), which was stirred until it dissolved. The mixture was cooled to 60° C. and more adipic acid (5 g) was added followed by Novozyme 435™ (0.78 g). The reaction was held for 2 hours at 2.63×10⁴ Pa (263 mbar). Further adipic acid (10 g) was added and after a further 2 hours at the same pressure the remaining adipic acid (16.5 g) was added. The material was left overnight, with stirring, before further Novozyme 435™ (0.71 g) was added. The pressure was then reduced to 6.6×10³ Pa (66 mbar) and the reaction held for a further 24 hours. Subsequently, the reaction was then held at 70° C. and a pressure of 1.3×10³ Pa (13 mbar) for 8 hours.

Molecular weight determination by GPC on the resulting polyurethane gave a weight average molecular weight (M_{w}) of 4500 and a dispersity of 2.4. (1000 Å column versus a polystyrene standard).

### Example 2A

| Materials | |
|---|---|
| Ethylene Carbonate | 28.23 g |
| 1,6-Hexamethylene diamine | 17.42 g |
| Toluene | 40 g |

Procedure

Ethylene carbonate was added to a flask and heated to 50° C., in an oil bath, followed by the addition of hexamethylene diamine (8 g). An exotherm to 75° C. followed and after 40 minutes the mixture solidified. Toluene (25 g) was added as an adjuvant and the oil bath temperature raised to 60° C. Further hexamethylene diamine (9.42 g) was added producing an exotherm to 85° C. The mixture again solidified and hot toluene (15 g) was added to triturate. The crystals were filtered off on cooling.

The material was crystallised twice from ethanol and dried to yield a biscarbamate in 70% yield and having a melting point of 94° C.

### Example 2B

| Materials | |
|---|---|
| Biscarbamate produced according to Example 2A | 7.25 g |
| 1,4-Butane diol | 22.72 g |
| Adipic acid | 40.17 g |
| Novozyme 435 ™ | 1.2 g |

Procedure

A biscarbamate produced according to Example 2A (7.25 g) and 1,4-butane diol (22.72 g) were transferred to a flange flask and the whole placed in an oil bath at 90° C. Adipic acid (8 g) was added under nitrogen, with stirring, and once dissolved, Novozyme 435™ (0.7 g) was added. The reaction was held at 5.26×10⁴ Pa (526 mbar); after 2 hours further adipic acid (25 g) was added. The oil bath temperature was adjusted to 60° C. and the process left overnight. The remaining adipic acid (7.17 g) was added and the pressure reduced to 1.31×10⁴ Pa (131 mbar). The material was left overnight at that pressure and then further Novozyme 435™ (0.5 g) was added. The pressure was reduced to 1.05×10⁴ Pa (105 mbar) and the bath temperature to 70° C. After about 8 hours the pressure was reduced to 6.6×10³ Pa (66 mbar) and the process again left to run overnight.

Molecular weight determination by GPC on the resulting polyurethane gave a weight average molecular weight Mw of 9350 and a dispersity of 1.75 (1000 Å column versus a polystyrene standard).

### Example 3

| Materials | |
|---|---|
| Ethylene carbonate | 35.3 g |
| 1,4-Butane diol | 36.0 g |
| 1,6-Hexamethylene diamine | 23.2 g |
| Adipic acid | 73.0 g |
| Novozyme 435 ™ | 2.42 g |

Procedure

Ethylene carbonate was added to a flask followed by 1,4-butane diol. The mixture was heated to 60° C. in an oil bath and hexamethylene diamine (6.0 g) was added. An exotherm to 70° C. developed; after 30 minutes further hexamethylene diamine (17.2 g) was added when a further exotherm to 88° C. was noted. The mix was left to stir overnight at 60° C. A clear liquid formed but rapidly crystallised on cooling to a white waxy solid.

Infra-red spectroscopy indicated the reaction had gone to completion and proved all the starting materials had been consumed.

The bath temperature was raised to 100° C., and an aliquot of adipic acid (30 g) was added to the biscarbamate/butanediol solution and the reaction stirred until the adipic acid had solubilized (about 0.5 hr). The mixture was cooled to 75° C., and the reaction rig changed over from reflux mode to distillation mode; Novozyme 435™ was added and the pressure reduced to 2.63×10⁴ Pa (263 mbar). After 1 hour, further adipic acid (15 g) was added. After 2 hours further adipic acid (15 g) was added and after a further 2 hours more adipic acid (13 g). The mixture was left overnight at 75° C. The pressure was then reduced to 1.05×10⁴ Pa (105 mbar) and after 1½ hours to 1.3×10³ Pa (13 mbar) the temperature of the oil bath was raised to 100° C. and processing continued for a further 24 hours under reduced pressure to produce a polyurethane of acid no. 0.7 mg KOH/g, and hydroxyl no. 77.5 mg KOH/g, corresponding to a molecular weight of 1488 daltons (close to the 1500 MW figure targeted). GPC assay gave Mₙ 2200, M_{w} 4641 and dispersity 2.11.

### Example 4A

| Materials | |
|---|---|
| Propylene carbonate | 51 g |
| Isophorone diamine | 42.5 g |

Procedure

Propylene carbonate was added to a flask, which was then transferred to an oil bath at 60° C. Isophorone diamine (10 g) was added under nitrogen. Further isophorone diamine (32.5 g) was added, portionwise, when an exotherm to 83° C. developed and the process was left overnight. NMR and GPC characteristics indicated that the reaction to biscarbamate had gone to completion.

### Example 4B

| Materials | |
|---|---|
| Biscarbamate produced according to Example 4A | 22.85 g |
| 1,4-Butane diol | 40 g |
| Adipic acid | 73.81 g |
| Novozyme 435 ™ | 2.5 g |

Procedure

1,4-Butane diol was added to a flange flask containing biscarbamate produced according to Example 4A and the flask was then transferred to an oil bath at 70° C. Adipic acid (30.15 g) was added with stirring. After dissolution, the oil bath temperature was reduced to 60° C. and Novozyme 435™ added. The mixture was heated at 60° C. for 4 hours at 5.26×10⁴ Pa (526 mbar) then adipic acid was added (43.66 g), with stirring. The pressure was then reduced to 2.63×10⁴ Pa (263 mbar) for 24 hours and then reduced further to 1.3×10³ Pa (13 mbar) and the process continue for a further 24 hours.

Molecular weight determination by GPC on the resulting polyurethane gave a weight average molecular weight (Mw) of 6000 and a dispersity of 2.14. (1000 Å column versus a polystyrene standard).

### Example 5A

| Materials | |
|---|---|
| Ethylene carbonate | 60.37 g |
| Bis(3-aminopropyl)polytetrahydrofuran (MW 350) | 120.1 g |

Procedure

Ethylene carbonate was melted out in a flange flask, by heating to 60° C. Bis(3-aminopropyl)PTHF was added, when an exotherm to 90° C. occurred. The reaction mixture was cooled to 60° C. and left at this temperature overnight, to give a reddish viscous liquid as product. NMR analysis showed that all the ethylene carbonate had been consumed; only a trace of starting amine was left.

### Example 5B

| Materials | |
|---|---|
| Biscarbamate from Example 5A | 70.32 g |
| Novozyme 435 ™ | 0.83 g |
| Adipic acid | 22.36 g |

The biscarbamate from Example 5A (70.32 g) was transferred to a flange flask and melted out at 60° C. Novozyme 435™ (0.5 g aliquot) was added followed by adipic acid (15.1 g) added in 3 sequential equal aliquots per hour over the next three hours. The pressure was reduced to 6.6×10³ Pa (66 mbar) and maintained for 12 hours and then reduced to 200 Pa (2 mbar) for a further 12 hours.

GPC analysis at this point indicated a product of MW 3000 daltons, but also indicated much unreacted starting material. Further adipic acid (7.2 g) was added, followed by Novozyme 435™ (0.33 g), the temperature of 60° C. was maintained, but the pressure was reduced to 6.6×10³ Pa (66 mbar) for 12 hours, before being decreased to 200 Pa (2 mbar) for a further 12 hours. At this point analysis showed that the light brown resin formed had acid no. 5.0 mg KOH/g and MW by GPC 6500 daltons.

### Example 6A

| Materials | |
|---|---|
| Ethylene carbonate | 41 g |
| Jeffamine D230 | 53.6 g |

Ethylene carbonate was heated to 60° C. in a flange flask and Jeffamine D230 (20.0 g) was added with stirring; a slight exotherm was noted. After 1 hour a further dose of Jeffamine (20.0 g) was added, again giving a slight exotherm. A third dose of Jeffamine (13.6 g) was added after cooling the flask down to 60° C. The flask was heated to 80° C. overnight giving a brown viscous liquid. TLC showed a single product; NMR indicated only a trace of remaining amine.

### Example 6B

Materials

| | |
|---|---|
| Biscarbamate from Example 6A | 30 g |
| 1,4-Butane diol | 6.7 g |
| Novozyme 435 ™ | 0.83 g |

Biscarbamate (30 g) from Example 6A and 1,4-butane diol was heated to 60° C. in a flange flask and adipic acid (21.7 g) was added in three equal portions over 3 hours. Pressure in the flask was reduced to 1.3×10⁴ Pa (131 mbar) and the process run for a further 48 hours to give a viscous brown polyester; GPC analysis indicated a MW 6500 daltons.

1. A process for producing a polyurethane, comprising polyesterifying at least one carbamate which is an aliphatic monocarbamate and/or an aliphatic or aromatic biscarbamate and/or an aliphatic or aromatic polycarbamate, said carbamate containing at least two functional groups selected from hydroxyl groups and carboxylic acid groups or esters or anhydrides thereof, in the presence of a polyesterification enzyme, and optionally in the presence of one or more copolymerizable monomers having two or more functional groups selected from hydroxyl groups and carboxylic acid groups or esters or anhydrides thereof.

2. A process according to claim 1, wherein the polyesterification is carried out in the presence of one or more monomers selected from aliphatic dicarboxylic acids or esters or anhydrides thereof, aliphatic hydroxycarboxylic acids or esters or anhydrides thereof, together with a diol or polyol.

3. A process according to claim 1 or claim 2, wherein the aliphatic monocarbamate is of general formula (I):
R¹—O—CO—NH—R² (I)
wherein
R¹ is a C₁ to C₁₂ hydroxyalkyl group optionally having one or more carbon-carbon double bonds and optionally having one or more carbon-carbon triple bonds, R¹ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl) and
R² is a C₁ to C₁₂ alkyl group optionally having one or more carbon-carbon double bonds and optionally having one or more carbon-carbon triple bonds, R² being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl) and, when substituted by hydroxyl, may be the same as or different from R¹;
the aliphatic or aromatic biscarbamate is of general formula (II):
R⁵—O—CO—NH—R—NH—CO—O—R⁶ (II)
wherein
R⁵ and R⁶, which may be identical or different, are C₁ to C₁₂ hydroxyalkyl groups optionally having one or more carbon-carbon double bonds and optionally having one or more carbon-carbon triple bonds, R⁵ and/or R⁶ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl) and
R is a single bond, an aromatic group, a cycloaliphatic group or a C₁ to C₁₂ alkylene group optionally having one or more carbon-carbon double bonds and optionally having one or more carbon-carbon triple bonds, R being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl); and
the aliphatic or aromatic polycarbamates include those of formula (IIA):
R^{a}(—NH—CO—O—R^{5a})ₘ (IIA)
wherein each R^{5a}, which may be identical or different, is a hydroxyalkyl group R⁵ as defined above for formula (II); R^{a} is an aromatic group, a cycloaliphatic group or an alkylene group R as defined above for formula (II);
and m is an integer of 3 or more.

4. A process according to claim 2 or claim 3, wherein the aliphatic dicarboxylic acid is of general formula (III):
HO₂C—R⁷—CO₂H (III)
wherein R⁷ is a bond or a C₁ to C₁₂ alkylene group optionally having one or more carbon-carbon double bonds and optionally having one or more carbon-carbon triple bonds, R⁷ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl); the aliphatic hydroxycarboxylic acid is of formula (IV):
HOCH₂—R⁸—CO₂H (IV)
wherein R⁸ is a bond or a C₁ to C₁₂ alkylene group optionally having one or more carbon-carbon double bonds and optionally having one or more carbon-carbon triple bonds, R⁸ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl); the aliphatic diol is of general formula (V):
HOCH₂—R⁹—CH₂OH (V)
wherein R⁹ is a bond or a C₁ to C₁₂ alkylene group optionally having one or more carbon-carbon double bonds and optionally having one or more carbon-carbon triple bonds, R⁹ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl); and the aliphatic polyol is of general formula (VI):
HOCH₂—R¹⁰—CH₂OH (VI)
wherein R¹⁰ is a C₁ to C₁₂ hydroxyalkylene group optionally having one or more carbon-carbon double bonds and optionally having one or more carbon-carbon triple bonds, R¹⁰ being optionally further substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl).

5. A process according to any one of the preceding claims, wherein the carbamate is prepared by reacting an aliphatic carbonate or hydroxy carboxylic acid or an ester or anhydride thereof, with a primary or secondary aminol, amino acid or ester thereof to produce a monocarbamate, with hydrazine or an aliphatic or aromatic diamine to produce a biscarbamate, or with an aliphatic or aromatic polyamine to produce a polycarbamate.

6. A process according to claim 5, wherein the aliphatic carbonate is of general formula (VII): 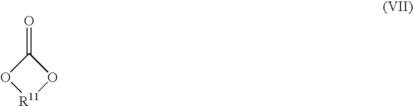
wherein R¹¹ is a C₂ to C₁₂ alkyl group optionally having one or more carbon-carbon double bonds and optionally having one or more carbon-carbon triple bonds, R¹¹ being unsubstituted or substituted with one or more substituents.

7. A process according to one of claims 5 or 6, wherein the aliphatic hydroxy carboxylic acid or ester or anhydride thereof is of general formula (VIII): 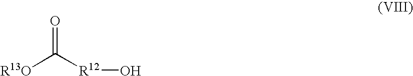
wherein R¹² is a C₂ to C₁₂ alkylene group optionally having one or more carbon-carbon double bonds and optionally having one or more carbon-carbon triple bonds, R¹² being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl) and R¹³ is hydrogen, or (CO)ₘR¹⁴ wherein m is 0 or 1 and R₁₄ is C₁ to C₁₂ alkyl.

8. A process according to any one of claims 5 to 7, wherein the primary or secondary aminol, amino acid or ester thereof is of general formula (IX):
HN(R¹⁵)—R¹⁶—X (IX)
wherein
R¹⁵ is a C₂ to C₁₂ alkyl group optionally having one or more carbon-carbon double bonds and optionally having one or more carbon-carbon triple bonds, R¹⁵ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl),
R¹⁶ is a C₂ to C₁₂ alkylene group optionally having one or more carbon-carbon double bonds and optionally having one or more carbon-carbon triple bonds, R¹⁶ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl) and
X is a hydroxyl, carboxylic acid or ester group.

9. A process according to any one of claims 5 to 8, wherein the aliphatic or aromatic diamine is of general formula (X):
HN(R¹⁸)—R^{17—NH(R}¹⁹) (X)
wherein
R¹⁷ is a bond, an aromatic group, a cycloaliphatic group or a C₁ to C₁₂ alkylene group optionally having one or more carbon-carbon double bonds and optionally having one or more carbon-carbon triple bonds, R¹⁷ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl),
R¹⁸ and R¹⁹ are the same or different and each is a C₁ to C₁₂ alkyl group optionally having one or more carbon-carbon double bonds and optionally having one or more carbon-carbon triple bonds, R¹⁸ and/or R¹⁹ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl).

10. A process according to any one of claims 5 to 9, wherein the aliphatic or aromatic polyamine is of general formula (XI):
R²⁰(—NHR²¹)ᵣ (XI)
wherein
R²⁰ is is an aromatic group, a cycloaliphatic group or a C₁ to C₁₂ alkylene group optionally having one or more carbon-carbon double bonds and optionally having one or more carbon-carbon triple bonds, R²⁰ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl)
each R²¹ is identical or different and is a C₁ to C₁₂ alkyl group optionally having one or more carbon-carbon double bonds and optionally having one or more carbon-carbon triple bonds, each R²¹ being unsubstituted or substituted with one or more substituents which may be the same or different, each substituent being selected from halogen, hydroxyl, OR³ (wherein R³ is C₁ to C₁₂ alkyl), carboxyl and —CO₂(CO)ₙR⁴ (wherein n is 0 or 1 and R⁴ is C₁ to C₁₂ alkyl); and
r is an integer of at least 3.

11. A polyurethane obtained by a process according to any one of claims 1 to 10.

12. Use of a polyurethane according to claim 11 as an adhesive, a textile coating, a surface coating, an elastomer, a thermoplastic polyurethane or a polyurethane dispersion.
